(19) United States
(12) Patent Application Publication
(10) Pub. No.: US 2025/0009532 A1
(43) Pub. Date: Jan. 9, 2025

(54) ADJUSTMENT VALVE

(71) Applicant: OTTO BOCK HEALTHCARE PRODUCTS GMBH, Wien (AT)

(72) Inventors: Andreas SCHUH, Wien (AT); Zsolt Tamas SZTRANCSIK, Wien (AT); Herman BOITEN, Wien (AT)

(21) Appl. No.: 18/748,524

(22) Filed: Jun. 20, 2024

(30) Foreign Application Priority Data
Jul. 4, 2023 (DE) 10 2023 117 664.1

(51) Int. Cl. A61F 2/74; A61F 2/50; A61F 2/60; F16K 27/06

(52) U.S. Cl. CPC A61F 2/748; A61F 2/60; F16K 27/065; A61F 2002/5006; A61F 2002/5033

(57) ABSTRACT

An adjustment valve for a hydraulic actuator, in particular for orthopaedic devices, in a flow duct, includes a valve sleeve and a valve unit. The valve sleeve has a sleeve wall which forms a receptacle space, and has at least one radially aligned sleeve wall opening. The valve unit is disposed within the receptacle space of the valve sleeve so as to be repositionable between a closed position and an open position. A cavity is formed by the valve unit. The valve unit has at least one valve unit wall which lies opposite the at least one sleeve wall opening and in which is formed a passage opening that in the open position lies opposite the corresponding sleeve wall opening. At least one duct is formed in the sleeve wall and/or the valve unit wall. Ducts formed in the sleeve wall open out at the sleeve wall opening, and ducts formed on the valve unit wall open out at the passage opening.

### FIELD OF THE INVENTION

The invention relates to an adjustment valve for a hydraulic actuator, in particular for orthopaedic devices, which is disposed in a flow duct and has an inlet and an outlet for a hydraulic fluid. The adjustment valve has a valve sleeve and a valve unit, the valve sleeve having a sleeve wall which forms a receptacle space, and at least one radially aligned sleeve wall opening being formed in the sleeve wall; the valve unit being disposed within the receptacle space of the valve sleeve so as to be repositionable between a closed position and an open position, and opening into a cavity formed by the valve unit; and having at least one valve unit wall which lies opposite the at least one sleeve wall opening and in which is formed a passage opening that in the open position lies opposite the corresponding sleeve wall opening. Depending on the flow direction, the cavity opens into an inlet or an outlet. If the hydraulic fluid flows from the valve sleeve into the cavity, the cavity opens into an outlet; conversely, if the hydraulic fluid flows into the cavity and through the passage opening and the sleeve wall opening, the cavity opens into the inlet.

### BACKGROUND

Orthopaedic devices are in particular but not exclusively prosthetics, orthotics and exoskeletons. Prosthetics replace limbs which are not or no longer present. Orthotics and exoskeletons are fixed to limbs and support, influence or delimit specific movements or positions of limbs relative to one another. In the case of orthotics or exoskeletons which extend across joints, an upper part is fixed to a proximal limb, and a lower part is fixed to a distal limb, said upper part and lower part being pivotably connected to one another by way of a joint.

Prosthetics can have a proximal upper part and a distal lower part, a joint device being disposed therebetween so as to achieve a pivotable connection between the upper part and the lower part.

Actuators or resistance devices, which influence a pivoting movement between a proximal upper part and a distal lower part of an orthopaedic device, can be disposed between the upper part and the lower part. The influence may take place by providing a resistance, for example by way of a hydraulic damper or pneumatic damper. A drive or a force accumulator for actively supporting a movement is assigned to the orthopaedic device, so as to actively effect a movement or so as to convert and store kinetic energy. As an alternative to joint devices, the orthopaedic devices can also influence a linear displacement of two components relative to one another.

In order to be able to influence the movement between two components, for example an upper part and a lower part of a joint device, devices for influencing the flow resistance are disposed in a flow duct of the hydraulic actuator, the latter potentially being designed as a hydraulic drive or a passive damper. Often, one or a plurality of adjustment valves by way of which the flow cross section can be adjusted is/are disposed in a flow duct between two hydraulic chambers that are separated by a piston. The adjustment can in particular take place based on sensors while the orthopaedic device is being used. For this purpose, sensor data is detected and forwarded to a control device. The sensor data is evaluated in the control device and converted into actuating signals during the use of the orthopaedic device, the adjustment valve being adjusted on the basis of said actuating signals. In this way, it is possible to adapt the orthopaedic device to the respective use scenario.

Owing to the comparatively small dimensions within orthopaedic devices, and the high pressures prevalent in the latter, high flow rates are achieved within the flow ducts and thus also within the adjustment valves. At times, this leads to high noise stress which can be perceived by the user of the orthopaedic device as well as to the environment. Moreover, hydraulic actuators in the regions of the adjustment valves are susceptible to flow losses, which leads to an increased basic resistance within the orthopaedic device. As a result, the precision in terms of control and the pivoting behaviour of the articulated components deteriorates, or the moving behaviour of the components between which the hydraulic actuator is disposed is permanently influenced, respectively. The flow rates that arise in the case of small dimensions of the flow ducts and high pressures at times cause turbulent flows and unwanted local temperature peaks and an unfavourable distribution of heat in the orthopaedic device. Temperature peaks cause an unwanted change in the viscosity of the hydraulic fluid.

### SUMMARY

It is an object of the present invention to provide an adjustment valve by way of which a more comfortable use and an improved control behaviour can be provided.

This object is achieved by an adjustment valve having the features described herein. Advantageous design embodiments and refinements of the invention are disclosed in the description and the figures.

The adjustment valve for a hydraulic actuator, in particular for orthopaedic devices, which is disposed in a flow duct and has an inlet and an outlet for a hydraulic fluid, having a valve sleeve and a valve unit; the valve sleeve having a sleeve wall which forms a receptacle space, at least one radially aligned sleeve wall opening being formed in the sleeve wall; the valve unit being disposed within the receptacle space of the valve sleeve so as to be repositionable between a closed position and an open position, and opening into a cavity formed by the valve unit and having at least one valve unit wall which lies opposite the at least one sleeve wall opening and in which is formed a passage opening that in the open position lies opposite the corresponding sleeve wall opening; is distinguished in that at least one duct is formed in the sleeve wall and/or the valve unit wall, and the duct formed in the sleeve wall opens out at the sleeve wall opening, and the duct formed on the valve unit wall opens out at the passage opening.

In the adjustment valve, hydraulic fluid flows, for example, radially from the outside through the initially one sleeve wall opening, and thus inlet opening, in the valve sleeve in the direction towards the valve unit, and in the case of a corresponding position of the valve unit relative to the valve sleeve, through the passage opening. A very precise flow which can be unequivocally influenced is easier to achieve in the case of laminar flows. The adjustment valve has the maximum flow of fluid when the sleeve wall opening and the passage opening are directly opposite one another. The flow cross section is reduced when the valve unit is repositioned, for example rotated or displaced, relative to the valve sleeve. By virtue of the at least one duct which is disposed or formed on the external side in the sleeve wall or the valve unit wall, the fluid is guided to the passage opening or sleeve wall opening via a path, wherein the flow characteristic of this path is laminar. As a result, in the case of a reduced flow cross section by virtue of the adjusted rotation or repositioning of the valve unit relative to the valve sleeve, it is possible to prevent excessive local heating in the region of the throttle point due to turbulent flows. Moreover, no noise, or only lesser noise, is caused by the laminar flows in comparison to turbulent flows, which renders the use of the orthopaedic device more comfortable for the user. A plurality of ducts can be formed in the sleeve wall and/or the valve unit wall and run in the direction towards the respective opening within the wall. These ducts can be of a rectilinear or in particular curved design. The duct cross section is in particular designed in such a way that there is a laminar flow. Accordingly, a rectilinear routing of the duct on the respective wall is advantageous, wherein the flow duct is naturally curved about the longitudinal extent in the case of a curved wall.

In one embodiment, a duct formed on the sleeve wall lies opposite a duct formed on the valve unit wall in such a way that the two ducts complement one another. The ducts can be disposed in such a way that they lie opposite one another on the entire adjustment path of the valve unit relative to the valve sleeve. Owing to the limited length of the ducts, the ducts are not completely congruent and complementary over the entire length. Alternatively, it is also possible and provided that there is congruence only over a sub-region of the length of the duct.

In one embodiment, the duct is at least partially formed as a duct or channel which is open towards the outside in such a way that, for example in the case of a semicircular cross section, a duct lying opposite complements the former so as to form a closed duct with a circular cross section. In the case of a smooth opposite wall, the duct or the channel is closed by the opposite wall. In the case of a duct in the sleeve wall, said duct is closed by the valve unit wall and vice versa in this instance.

In one embodiment, the duct is enlarged in the direction towards the passage opening, or sleeve wall opening, respectively, and causes a reduction in the flow rate in the direction towards the opening that directs the hydraulic fluid to an outlet.

In one embodiment, with the exception of the at least one passage opening and an access opening, the cavity is of a closed design, wherein the end wall of the cavity that lies opposite the access opening is in particular closed. A plurality of passage openings can be formed within the valve unit wall so as to correspond to sleeve wall openings in the sleeve wall, so as to be able to direct a plurality of partial flows of the hydraulic fluid into the cavity or out of the latter. The otherwise closed embodiment of the cavity promotes the effective deflection and influencing of the flow rates of the hydraulic fluid.

In one embodiment, the valve unit is mounted so as to be rotatable or displaceable in the valve sleeve. Different cross sections for the hydraulic fluid in the cavity of the valve unit are provided by rotating or displacing the valve unit in the valve sleeve. The total available inlet cross section results from the intersecting geometries in the sleeve wall opening and the respectively opposite passage opening. In the case of a static valve sleeve and a valve unit which is mounted so as to be movable relative thereto, the inlet cross section can be enlarged or reduced, and completely closed in an extreme case, by the relative movement. It is possible to generate an optimal cross section for the inflow opening for each rotation angle, or for each displacement position, by way of a corresponding geometry of the sleeve wall opening as well as of the passage opening. For example, proceeding from a closed position, it can be expedient and desirable to initially cause only a slow enlargement of the inlet cross section, and to permit the enlargement to increase more progressively only after a predetermined rotation angle, or after a displacement position, so as to then reduce the gradient of enlargement up to the complete open position in order to enable fine adjustment. Conversely, the variation per unit of the displacement path, or per adjustment angle, can also be chosen to be small in a central opening range, in order to enable fine adjustment in a central damping range or activation range.

In one embodiment, the valve unit is coupled to an adjustment device by way of which it is possible to adjust, or move, the valve unit to the respectively desired position, and to keep the valve unit in the respectively desired position. The adjustment device can be coupled to a manual or motorized drive; the adjustment device can also be coupled to a mechanical drive, for example to cause opening or closing of the inlet cross section as a function of travel or load.

The valve sleeve can be formed as part of a housing of the hydraulic actuator, for example as part of a damper housing. Alternatively, the valve sleeve is formed as a separate component in such a way that an adjustment valve can be formed and separately made as a modular unit from the valve sleeve, valve unit and diffusor insert. Such an adjustment valve can then be inserted into the hydraulic actuator and optionally be coupled to the adjustment device.

In one embodiment, the sleeve wall opening and/or the passage opening are/is designed as a slot or bore, wherein a plurality of openings can be disposed or formed in the respective walls, so as to enable a hydraulic fluid to pass through. For example, a plurality of correspondingly disposed and aligned bores can lie opposite a slot in such a way that hydraulic fluid enters one slot from two bores, and vice versa. For example, the slot is formed in the valve unit, while the bores are formed next to one another in the sleeve wall.

In one embodiment, the sleeve wall opening and/or the passage opening are/is designed as a slot and oriented obliquely in relation to the longitudinal extent of the adjustment valve. The slot can be designed to be rectilinear, thus having a constant gradient, on the circumference of the valve sleeve and/or the valve unit. Alternatively, the slot is curved, or curved at least in regions, as a result of which different intersections of the sleeve wall opening and the passage opening can be achieved in a uniform adjustment movement. The width of the slot can vary along its longitudinal extent so as to achieve an enlargement or reduction in the width along its longitudinal extent, or along the axial extent of the adjustment valve. An adapted variation of the inlet cross section into the cavity can be achieved as a result.

In one embodiment, the ducts are disposed or formed in such a way that in the closed position there is no fluidic connection between the sleeve wall opening and the passage opening. Also, no hydraulic fluid can pass through the ducts in the closed position. This is achieved, for example, in that in the case of a rotatable mounting of the valve unit in the valve sleeve, the length of the duct is designed in such a way that the latter terminates in front of the passage opening, or sleeve wall opening, when the adjustment valve is in the closed position. For example, if only one sleeve wall opening and one passage opening are disposed across the circumference of the adjustment valve in the case of a rotatable embodiment, the maximum circumferential length of the duct is the circumference of the wall in which the duct is formed, minus the width of the sleeve wall opening and the width of the passage opening. In the case of a displaceable mounting along the longitudinal extent, the length of the duct is the maximum displacement length, minus the dimension of the opposite opening. The interruption of the fluidic connection can also take place by a corresponding curvature and a corresponding profile of the duct on the surface of the sleeve wall or valve unit wall. The ducts do not cover one another in the closed position, but absolute sealing is not possible owing to the usual manufacturing tolerances and the annular gap between the valve unit and the valve sleeve. However, in the application there is almost no volumetric flow worth mentioning in the closed position.

In one embodiment, the cavity opens into an outlet, and a diffusor insert which deflects the hydraulic fluid from the passage opening in the direction towards the outlet is disposed in the cavity. It is prevented by the diffusor insert that the radially inflowing hydraulic fluid impacts the opposite wall of the valve unit, or mixes with a hydraulic flow entering from a further sleeve wall opening, or impacts said hydraulic flow. This would cause turbulent flows within the cavity within the valve unit and thus high noise stress and an increased flow resistance, in particular in the case of completely opened passage openings. The diffusor insert within the cavity of the valve unit deflects the hydraulic fluid in such a way that it is deflected in the direction towards the outlet, instead of impacting orthogonally a valve unit wall. It is avoided as a result that voids and turbulences are formed within the cavity of the valve unit, so that the flow resistance and the generation of noise are reduced.

In one embodiment, the diffusor insert is made separately from the valve unit and fastened to or in the valve unit. As a result, it is possible to produce the diffusor insert from a different material than the valve unit, in particular from a different material than the valve unit wall in which the sleeve wall opening or the sleeve wall openings is/are formed. Moreover, the separate production of the diffusor insert enables the external contour or geometry of the diffusor insert to be adapted to different use conditions or models, as well as to different hydraulic actuators. Moreover, manufacturing complex three-dimensional structures that are freely accessible from the outside is easier than within a sleeve-type valve unit. Alternatively, the diffusor insert is an integral constituent part of the valve unit and produced in the context of an additive manufacturing method, for example, in which complex three-dimensional structures can be produced during the primary shaping method by a layered construction of material layers.

In one embodiment, the diffusor insert is designed to taper in the direction towards the outlet, so as to gradually enable an enlargement of the flow cross section in the direction towards the outlet.

In one embodiment, the valve unit has a plurality of passage openings; the diffusor insert has partition walls which are disposed between the passage openings. As a result, it is possible to separate the flow ducts of the respective passage openings and to individually deflect said flow ducts. Different flow conditions can prevail within flow ducts, so that the deflection of the partial flows can also take place in different ways in order to achieve optimal influencing and deflecting of the partial flows.

In one embodiment, the partition walls do not extend up to the end of the cavity or the outlet, but sub-divide the cavity of the valve unit only over a partial length in the axial extent in such a way that the individual partial flows of the hydraulic fluid are still converged within the cavity. It is advantageous herein for the partition walls to taper in the direction towards the outlet in such a way that this results in a gradual widening of the cross section for the fluid flow in order to maintain or generate as little turbulent flow as possible, in particular in order to maintain or generate a laminar flow. In one alternative, the partition walls extend up to the outlet in such a way that the individual partial flows are unified only after the outlet.

An inlet to the adjustment valve and an outlet from the adjustment valve can be fluidically connected to a piston/cylinder unit of the orthopaedic device and be connected to two chambers that are separated by the piston of the piston/cylinder unit. A hydraulic circuit between the inlet and the outlet is established as a result. In principle, it is possible that there is only one flow duct between the chambers of the piston/cylinder unit in which the adjustment valve is disposed. In this instance, the adjustment valve, optionally with the diffusor insert, is effective in only one direction. In the opposite direction, the inlet would then be the outlet, and the hydraulic fluid would flow at a high pressure through the cavity and the passage openings back into the sleeve wall opening. A plurality of adjustment valves are preferably disposed in the hydraulic connections between the chambers, in particular so as to enable separate influencing of the adjustment movements.

In one embodiment, the duct or the channel or channels, respectively, is/are embodied in such a manner that a laminar flow is formed in the duct and there is a proportional correlation between pressure and volumetric flow. This is achieved in particular in that the duct has a constant width and/or a constant depth.

The invention likewise relates to a hydraulic actuator having an adjustment valve described above.

### DESCRIPTION OF THE DRAWINGS

Exemplary embodiments of the invention will be explained in more detail hereunder by means of the appended figures in which:

FIG. 1 shows a schematic illustration of a worn prosthetic;

FIG. 2 shows a schematic illustration of a prosthetic leg;

FIG. 3 shows a sectional illustration of an adjustment valve;

FIG. 4 shows an individual illustration of a diffusor insert;

FIG. 5 shows a sectional illustration of an adjustment valve assembly;

FIG. 6 shows a perspective illustration of a diffusor insert;

FIG. 7 shows an oblique rear view of the diffusor insert;

FIG. 8 shows a sectional illustration through a valve unit having a diffusor insert;

FIG. 9 shows an exploded illustration of an adjustment valve;

FIG. 10 shows the adjustment valve according to FIG. 9 in an open position;

FIG. 11 shows a perspective illustration of the adjustment valve according to FIG. 10;

FIG. 12 shows a longitudinal sectional illustration through an adjustment valve;

FIG. 13 shows a cross-sectional illustration through an open adjustment valve;

FIG. 14 shows a perspective illustration of a closed adjustment valve;

FIG. 15 shows sectional illustrations through the adjustment valve according to FIG. 14;

FIG. 16 shows an adjustment valve in a perspective illustration;

FIG. 17 shows the adjustment valve according to FIG. 16 in a partially transparent illustration;

FIG. 18 shows the adjustment valve according to FIG. 17 in a partially closed illustration;

FIG. 19 shows an illustration of the flow profile in the adjustment valve according to FIG. 18;

FIG. 20 shows the adjustment valve according to FIG. 16 in a sectional illustration;

FIG. 21 shows the adjustment valve with duct; and

FIG. 22 shows a valve unit in an individual illustration.

### DETAILED DESCRIPTION

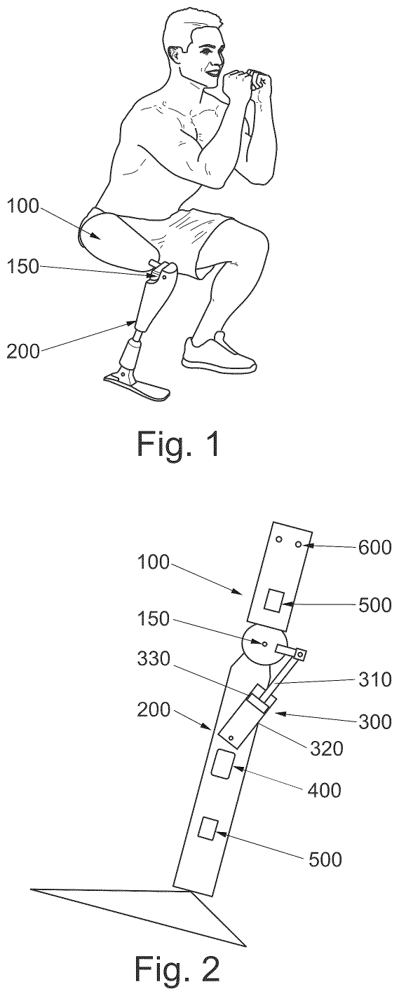

Shown in FIG. 1 is a person using an orthopaedic joint device of a lower extremity, having an upper part 100 which has a prosthetic shaft, and a lower part 200 in the form of a shank part having a prosthetic foot fastened to the distal end. The upper part 100 is pivotable relative to the lower part 200 about a pivot axis 150.

Illustrated in a schematic illustration in FIG. 2 is an orthopaedic joint device in the form of a prosthetic of the lower extremity. The orthopaedic joint device has an upper part 100 and a lower part 200, which are mounted on one another so as to be pivotable about a pivot axis 150. An actuator 300, which in the exemplary embodiment illustrated is designed as a hydraulic damper, is disposed on the upper part 100 and on the lower part 200, so as to influence the relative pivoting of the upper part 100 in relation to the lower part 200 about the pivot axis 150. The hydraulic damper 300 is formed as a piston/cylinder unit and in a housing has a cylinder which is divided into two hydraulic chambers 310, 320 by a piston 330 that is coupled to a piston rod. At least one fluidic connection is formed between the two hydraulic chambers 310, 320, so that hydraulic fluid can flow into the one chamber when the latter is enlarged, and conversely can flow out of the chamber when the latter is reduced and flow into the opposite chamber. An adjustment valve which will be described in more detail hereunder is disposed in the fluidic connection—not illustrated—between the two chambers 310, 320. In alternative embodiments, the actuator 300 can be equipped as an active actuator having a drive, for example an electric motor, a force accumulator, or any other type of drive, so as to influence a pivoting movement of the upper part 100 relative to the lower part 200. In the exemplary embodiment illustrated, the actuator 300 is designed as a passive actuator and provides a resistance in relation to a flexion motion and optionally an extension motion. The actuator 300 is coupled to a control device 400 which in the exemplary embodiment illustrated is disposed on the lower part 200. The control device 400 is coupled to sensors 500 which are disposed on the upper part 100 and/or the lower part 200. The sensors 500 detect state variables of the joint device, for example positions, locations, forces, momentums, accelerations or orientations of components in space or relative to one another, wherein a multiplicity of sensors can be used in order to detect the desired state variables of the orthopaedic joint device. The sensors 500 are coupled to the control device 400 and transmit corresponding sensor data or sensor signals to the control device 400, wherein the actuator 300 is varied in terms of its capability to influence the pivoting movement or pivotability of the upper part 100 relative to the lower part 200, based on the sensor values or sensor data. For example, the flexion resistance and/or the extension resistance are/is varied as a function of the sensor values. Valves or throttles are adjusted for this purpose. In alternative embodiments, magnetic fields can be varied in order to vary magnetorheological fluids in terms of their viscosity. In the case of a mechanical brake device, braking forces can be increased or reduced in order to generate an adapted resistance behaviour of the actuator 300. In the case of an active drive which has at least one electromechanical actuator, resistances in relation to a movement can be applied via currents and voltages, or else movements can be supported by the latter. Apart from applying momentums and momentum profiles, trajectories can be tracked, or system characteristics in the sense of impedance or admittance control can be emulated, by way of control algorithms and sensor information pertaining to the pivoting movement of the upper part 100 and the lower part 200. For example, it is possible to emulate the behaviour of a linear or non-linear spring, the behaviour of a damper, or an inertia, or a combination of a plurality of characteristics, as a result of which a pivoting movement counter to a flexion can be influenced. Such an actuation offers a high degree of flexibility. Pivoting movements can also be actively supported by controls of this type. If a momentum or a force is applied by way of an actuator without pivoting taking place, for example because there is an equilibrium of external and internal forces, a resistance is likewise offered to the pivoting movement. Energy stores, for example a hydraulic spring accumulator, can also be activated and deactivated, gear ratios of drives can be varied, and/or the latter be engaged or disengaged, by way of actuators. These types of actuations can likewise be applied in order to influence the pivoting movement. Resistances are forces and momentums which are applied by actuators in order to influence a pivoting movement. A resistance may be counter to a pivoting movement, or else support a pivoting movement, i.e. act in the direction of the pivoting movement.

Biosignal sensors 600 which are designed to detect muscular activity or actuate a muscle or the musculature, and generate biosignals which are transmitted to the control device 400, are formed on the upper part 100, or fastened thereto, for example on a prosthetic shaft or on a mount. The biosignal sensors 600, which can be designed as discharge electrodes, are coupled to the control device 400 by way of a conducting connection or wirelessly, and transmit corresponding signals to the control device 400 which likewise activates, deactivates or modulates the actuator 300 based on the biosignals in order to vary the resistance, or to influence the movement behaviour or the repositionability of the components relative to one another.

Deviating from the embodiment of the orthopaedic joint device as a prosthetic, said orthopaedic joint device can also be designed as orthotic, wherein a shank brace on an artificial knee joint is formed as a lower part 200 instead of the shank part. In this instance, the upper part 100 is a thigh brace which can be fixed to the thigh by way of corresponding fastening devices such as belts, shells or sleeves. The biosignal sensors 600 are either fastened separately to the thigh or any other musculature group, and can also be disposed on the fastening device. The orthopaedic joint device can also be designed as an exoskeleton, as a special form of an orthotic. As an alternative to the illustrated embodiment as an orthopaedic joint device of the lower extremity, the orthopaedic joint device can also be designed for an upper extremity, for example as an artificial shoulder joint or an artificial elbow joint for a prosthetic arm, or as an orthotic or an exoskeleton for an arm, respectively.

Activating, deactivating and optionally modulating the actuator for influencing a pivoting movement or the pivotability of the joint device take place on the basis of at least one signal from a sensor in such a manner that the influencing of the pivoting movement, or pivotability, in relation to flexion is increased when a corresponding sensor signal is present. Influencing the pivoting movement is also understood to mean that a pivoting movement is blocked in such a way that the lower part 200 is locked relative to the upper part 100, and no relative movement between the two parts takes place and can take place. Influencing the pivotability of the upper part 100 relative to the lower part 200 can thus be performed by blocking the joint in the direction of flexion, or by increasing the resistance to flexion. Influencing is carried out either by a passive or an active actuator. In order to reduce the increased resistance of the joint device to flexion again, this takes place based on sensor values, thus on values which are transmitted to the control device 400 by the sensor 500 or the sensors 500 and/or biosignal sensors 600. Controlling the orthopaedic joint device, for example of a lower extremity, in such a way is advantageous and expedient for a stand-up function, for example, in which flexion, for example of the knee joint, is to be impeded or blocked. Influencing the pivotability takes place by an adjustment of an adjustment valve, for example.

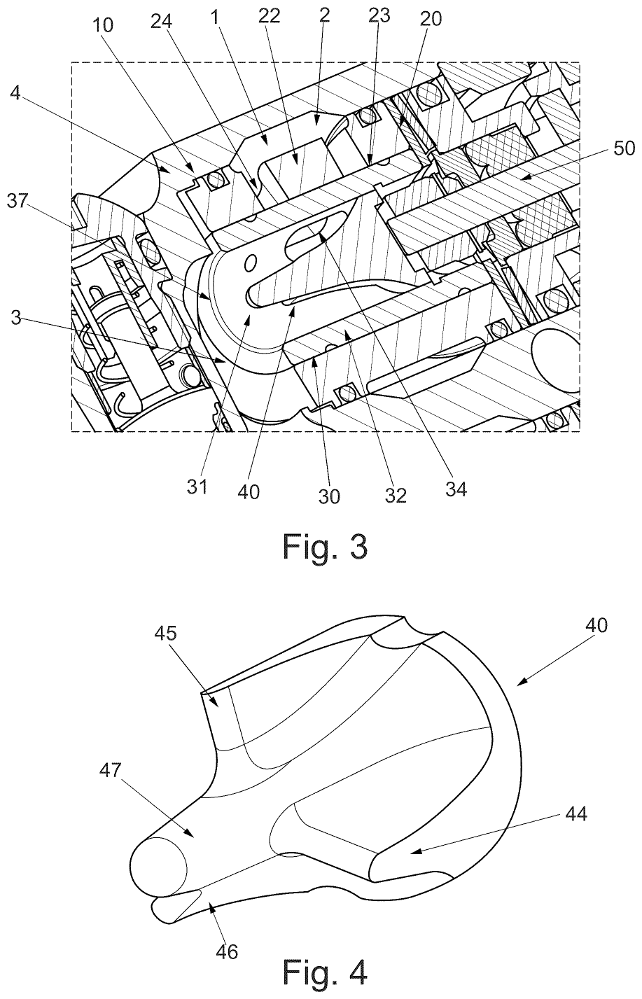

Illustrated in a sectional illustration in FIG. 3 is an adjustment valve 10 which is disposed in a flow duct 1 which is located between one of the two chambers 310, 320, the latter not being illustrated. Hydraulic fluid within the flow duct is transported to an inlet 2 of the adjustment valve 10. As soon as the flow of the hydraulic fluid has passed through the adjustment valve 10, said hydraulic fluid exits the latter through an outlet 3. The adjustment valve 10 has a valve sleeve 20 which in the exemplary embodiment illustrated is of a substantially cylindrical design. The valve sleeve 20 has a sleeve wall 22 which surrounds a receptacle space 23. A valve unit 30 is disposed within the receptacle space 23. Hydraulic fluid is directed from the inlet 2 out of the flow duct 1 through sleeve wall openings 24 in the direction towards the valve unit 30. Only one sleeve wall opening 24 is illustrated in the sectional illustration of FIG. 3, which is divided into two partial inlet openings, assuming that the hydraulic fluid flows from the outside to the inside; conversely, these would be partial outlet openings. The sleeve wall opening 24 is radially aligned in such a way that hydraulic fluid which is transported from the outside through the flow duct 1, which circumferentially surrounds the valve sleeve 20, to the sleeve wall opening 24, can make its way into the interior of the valve sleeve 20. The flow duct 1 is disposed in an annular manner about the valve sleeve 20, so that the hydraulic fluid can be directed in the direction towards the valve unit 30 through a plurality of sleeve wall openings.

The valve unit 30 is mounted so as to be repositionable within the receptacle space 23 of the valve sleeve 20, in the exemplary embodiment illustrated mounted so as to be rotatable. By rotating the valve unit 30 within the valve sleeve 20 it is possible to open and close the adjustment valve 10. For this purpose, the valve unit 30 is rotated between a closed position and an open position. The adjustment valve 10 is partially open, or partially closed, in the intermediate positions, respectively, so that a reduced volumetric flow of the hydraulic fluid is established. Associated therewith is an increased flow resistance in the hydraulic system so that the resistance of the hydraulic actuator can be adjusted. In order to allow the hydraulic fluid to pass from the inlet 2 to the outlet 3 through the sleeve wall opening 24, the valve unit 30 has at least one valve unit wall 32 which lies opposite the sleeve wall opening 24, or the sleeve wall openings, and in which is formed at least one passage opening 34 which in an open position lies opposite the corresponding or opposite sleeve wall opening 24. Owing to the sleeve wall opening 24 and the passage opening 34 being superimposed in the open position, a maximum cross section of the passage opening for the hydraulic fluid is established. The cross section of the passage opening is 0 in the closed position, so that no hydraulic fluid can pass from the inlet 2 to the outlet 3. Different passage cross sections are generated by rotating the valve unit 30 within the receptacle space 23, so as to adjust the desired hydraulic resistance.

As an alternative to rotation, which is illustrated in FIG. 3, the variation of the passage cross section can also take place by displacing the valve unit 30 relative to the valve sleeve 20. The rotation of the valve unit 30 takes place about a rotation axis which also defines the longitudinal extent of the adjustment valve 10; when displacing the valve unit 30 within the valve sleeve 20, the displacement direction is the longitudinal extent of the adjustment valve 10.

Two annular seals 25 which cause sealing in relation to a housing or the hydraulic line on both sides of the flow duct 20 are disposed on the external circumference of the valve sleeve 20 in order to prevent leakage. An adjustment device 50 by way of which the valve unit can be rotated is disposed on the valve unit 30 for rotating or repositioning the valve unit 30 relative to the valve sleeve 20. The adjustment device 50 in the exemplary embodiment illustrated is designed as a shaft which is coupled to a drive or a manually activatable actuating element or actuating wheel. The adjustment can take place manually or by way of a motorized drive, the latter preferably being controlled based on sensors by a control device so as to cause the hydraulic resistance to be adapted on the basis of sensor data.

Formed within the valve unit 30 is a cavity 31 which in the exemplary embodiment illustrated is of a cylindrical design. Disposed within the cavity 31 is a diffusor insert 40 which is disposed in a rotationally fixed manner within the cavity 31 and so as not to be repositionable in the longitudinal extent of the valve unit 30. That end of the valve unit 30 that lies opposite the outlet 3 is sealed, in particular sealed by the diffusor insert 40. For this purpose, the diffusor insert 40 has a substantially cylindrical end piece which is in an exact fit within the cavity 31. The cylindrical end piece almost reaches up to the passage openings 34 which in the exemplary embodiment illustrated lie on the same circumferential region which, like the sleeve wall openings 24, is located between the annular seals. The diffusor insert 40 in the exemplary embodiment illustrated is formed as a separate component which is inserted and fixed within the valve unit 30. As an alternative to a design in multiple pieces, the diffusor insert 40 can also be formed integrally with the valve unit 30, either produced by an additive manufacturing method or by a corresponding cutting method from a solid material.

The diffusor insert 40, proceeding from its rear end that closes the cavity 31, tapers in the direction towards the outlet 30 and has a radiused shape in such a way that hydraulic fluid which flows radially from the outside through the sleeve wall opening 24 and the passage opening 34 is diverted by 90° in the direction towards an access opening 37, presently an outlet opening 37 of the cavity 31, by the diffusor insert. Owing to the altered shape of the surface of the diffusor insert 31 in the direction towards the outlet opening 37, the flow cross section of the region of the cavity 31 that is formed between the internal wall of the valve unit 30 and the diffusor insert 40 is enlarged. Owing to the radiused surface shape having the curvature in the direction towards the outlet opening 37, and the gradual enlargement of the flow cross section, a deflection of the flow direction and a deceleration of the flow rate take place in such a way that turbulences within the hydraulic fluid are avoided or at least reduced.

The adjustment valve 10 has a plurality of passage openings 34 and sleeve wall openings 24 which are disposed, preferably so as to be identically spaced apart, radially about the valve sleeve 20 and the valve unit 30. As a result, it is possible to allow an overall comparatively large flow of the hydraulic fluid through a multiplicity of passage openings 34 into the cavity 31. In such an embodiment with a plurality of sleeve wall openings 24 and passage openings 34 it is advantageous to dispose partition walls 44, 45, 46 in the region of the passage openings 34, said partition walls 44, 45, 46 being illustrated in the perspective illustration of the diffusor insert 40 in FIG. 4. The diffusor insert 40 in FIG. 4 is an integrally designed formed part which has three partition walls 44, 45, 46 which in the installed state within the valve unit 30 reach up to the internal wall of the cavity 31. In the case of a cylindrical embodiment of the cavity 31, the partition walls 44, 45, 46 extend rectilinearly from the rear end of the diffusor insert 40 in the direction towards the outlet 3, or the access opening 37 formed as an outlet opening, respectively. Trough-shaped, radiused flow surfaces which from the external circumference of the valve unit 40 extend radially inwards in the direction towards a central member 47 are in each case formed between the rear walls 44, 45, 46. The central member 47 is of a conical design and tapers in the direction towards the outlet opening 37. The central member 47 advantageously terminates in front of the outlet opening 37, so as to provide the entire cross section of the cavity 31 in the region of the outlet opening 37 as a flow cross section for the hydraulic fluid. The partition walls 44, 45, 46 terminate in front of the tip of the central member 47, wherein a radiused transition results between the troughs between the partition walls 44, 45, 46 as well as radially towards the inside, so as to avoid turbulences within the hydraulic fluid.

A recess is formed as an anti-rotation safeguard on the rear end of the diffusor insert 40, said recess being able to engage in a form-fitting manner in a projection within the valve unit 30.

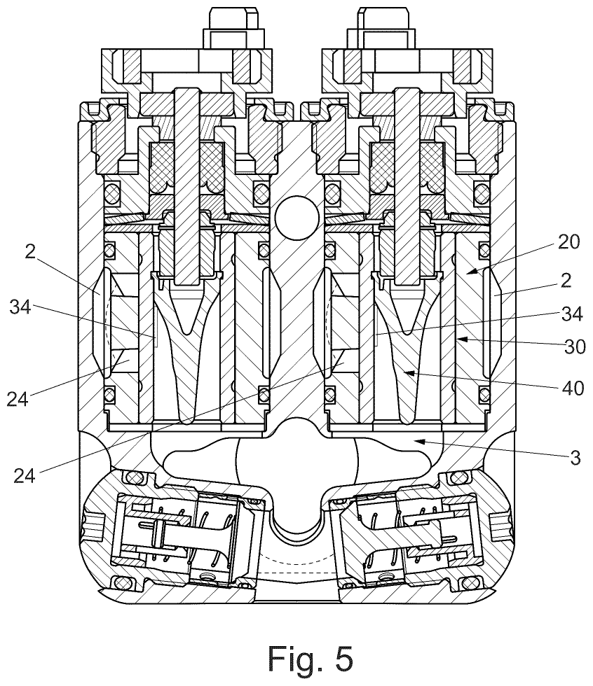

Illustrated in FIG. 5 is a cross-sectional illustration of two adjustment valves 10 which are disposed so as to be mutually parallel and open into a common outlet 3. From the outlet 3, the hydraulic fluid can be transported to the respective chamber of the hydraulic actuator by check valves. In principle, it is also possible to allow a movement of the hydraulic fluid in the opposite direction without check valves.

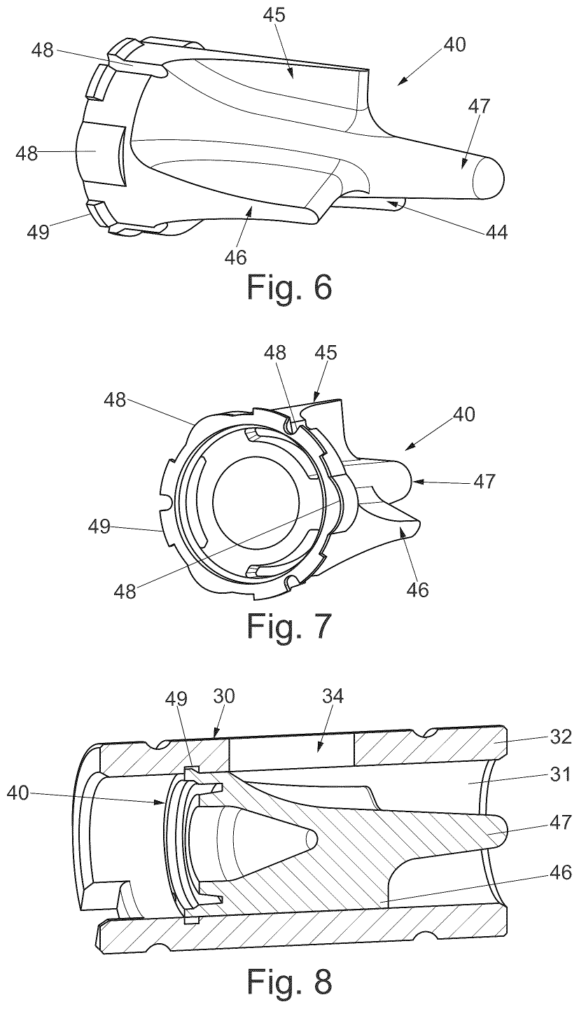

A variant of the diffusor insert 40 is illustrated in FIG. 6. This diffusor insert 40 also has three partition walls 44, 45, 46 which are mutually oriented so as to be identically distributed across the circumference in such a way that the central planes of the partition walls 44, 45, 46 are mutually oriented at an angle of 120°. Anti-rotation safeguards 48 and likewise axial safeguards 49, which prevent the diffusor insert 40 being displaced and rotated within the valve unit 30, are formed on the rearward end of the diffusor insert 40.

The diffusor insert 40 according to FIG. 6 is illustrated in a rear view in FIG. 7. The anti-rotation safeguards 48 and the axial safeguards 49 on the rearward end, and likewise the embodiment as a hollow member, and the flow surface which extends in a curvature towards the front, in the direction towards the tip of the central member 47 in such a way that the diffusor insert 40 tapers in the flow direction from the inlet 2 to the outlet 3, can be seen. The partition walls 44, 45, 46 also taper in the direction towards the tip of the central member 47, preferably by way of a curved contour.

FIG. 8 shows a cross-sectional view of the valve unit 30 having the inserted diffusor insert 40 according to FIGS. 6 and 7. The external circumference of the partition walls here bears on the internal wall of the valve unit 30; the partition wall 46 bearing on the internal wall of the cavity 31 is illustrated. The passage opening 34 is disposed within the valve unit wall 32 so as to be between the two other partition walls in such a way that three mutually separate inflow regions are formed by the partition walls 44, 45, 46 within the cavity 31. The hydraulic fluid flow which flows radially from the outside through the passage openings 34 is deflected by 90° on the surface of the diffusor insert 40 and directed along the tapered central member 47 in the direction towards the outlet opening.

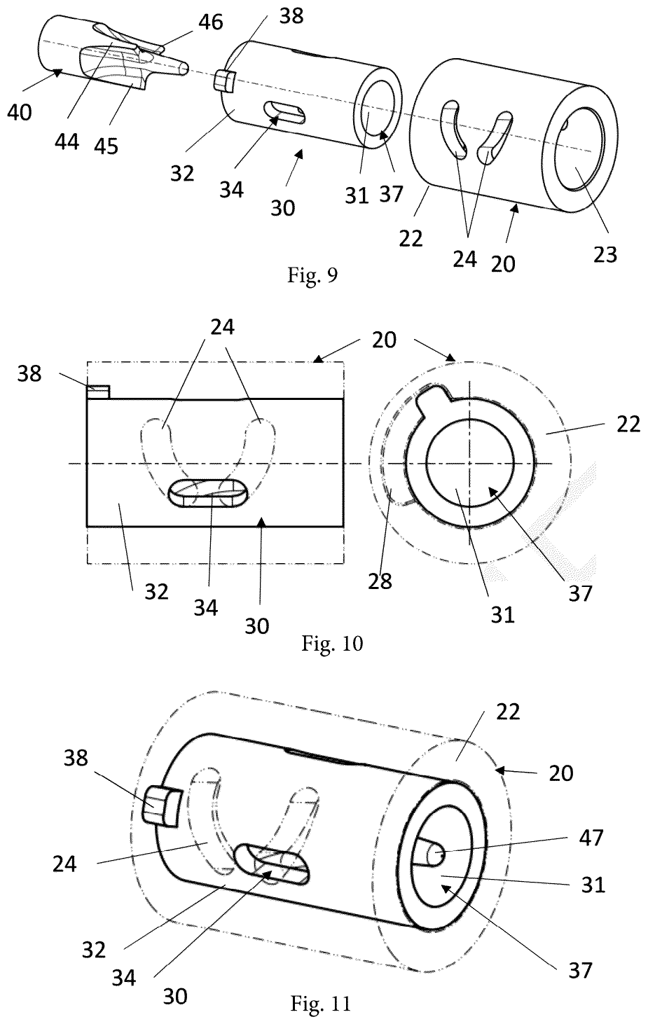

The adjustment valve having the three main components valve sleeve 20, valve unit 30 and diffusor insert 40 is illustrated in an exploded illustration in FIG. 9. The valve sleeve 20 is of a cylindrical design and has a substantially closed sleeve wall 22 in which sleeve wall openings 24 are uniformly disposed so as to be offset by 120° about the circumference. In the exemplary embodiment illustrated, the sleeve wall openings 24 are formed by two partial openings which are designed as curved slots. The slots are oriented so as to be inclined in relation to the longitudinal extent of the valve sleeve 20; alternatively, said slots could also be designed to be rectilinear and oriented obliquely in relation to the longitudinal extent. Within the valve sleeve 20, the receptacle space 23 is formed as a cylindrical shell and receptacle space for the valve unit 30. The sleeve wall openings 24 penetrate the sleeve wall 22 in such a way that fluid can pass through the sleeve wall opening 24 in the direction towards the receptacle space 23 and to the valve unit 30 through the sleeve wall 22.

In the embodiment illustrated, the valve unit 30 likewise has a cylindrical external circumference. The valve unit 30 is formed as a sleeve-type insert by the valve unit wall 32, in which three passage openings in the sleeve wall 32 are disposed behind one another across the circumference so as to be offset by 120° in the circumferential direction. The passage openings, of which only one passage opening 34 can be seen, are formed as a rectilinear slot in the exemplary embodiment illustrated, said slot extending along the longitudinal extent of the valve unit 30. The valve unit 30 forms the cavity 31 having the access opening 37. A form-fit element 38, as a detent limit to restrict rotation within the valve sleeve 20, is disposed on the external side of the valve unit 30 on the rearward end that lies opposite the access opening 37.

The diffusor insert 40 is designed having three partition walls 44, 45, 46, in a manner analogous to the previously described diffusor inserts, and is disposed so as to be rotationally fixed and axially immovable within the cavity 31 of the valve unit 30. All components of the adjustment valve 10 are coaxially oriented and disposed in one another. As an alternative to an arrangement of the passage opening 34 as a rectilinear slot along the longitudinal extent of the valve unit 30, said passage opening 34 can also be formed on the circumference of the valve unit wall 33 so as to be inclined in relation to the longitudinal extent. The embodiment can be designed in a manner similar to the passage openings 34 on the sleeve wall 22 of the valve sleeve 20. Likewise, the sleeve wall opening 24 can be designed as a longitudinally oriented slot. It is likewise possible that both openings, the sleeve wall opening 24 as well as the passage opening 34, are designed like the sleeve wall opening 24 illustrated in FIG. 9, wherein the dimensions, shapes and orientations have to be designed in such a way that there are no overlaps of the openings whatsoever for completely closing the adjustment valve. In order to be moved to the open position, the valve unit 30 is rotated relative to the valve sleeve 20 until a maximum congruence of the openings is achieved.

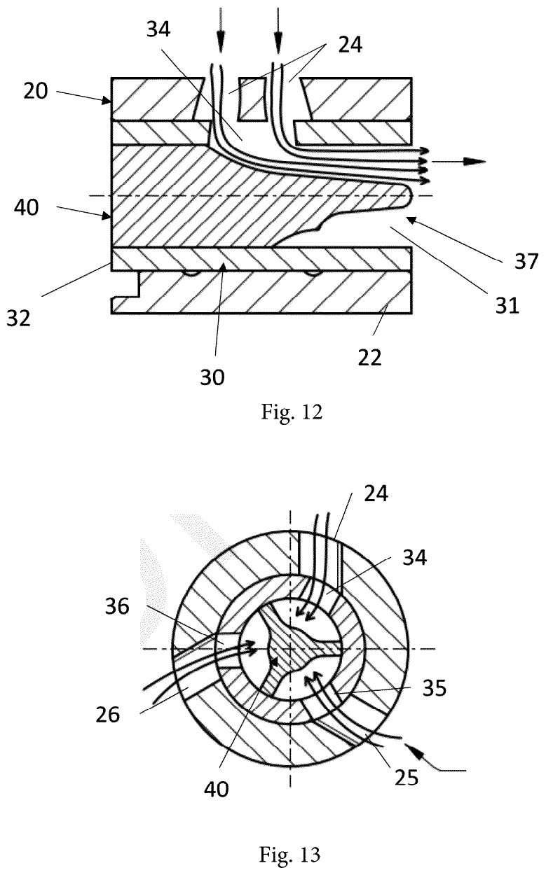

The adjustment valve is illustrated in the open position in FIG. 10 as well as in FIG. 11. FIG. 10 shows a longitudinal section and a cross section, wherein the valve sleeve 20 is illustrated by dashed lines. FIG. 11 shows a perspective view having a maximum passage opening which is achieved by the sleeve wall opening 24 being congruent with the passage opening 34 across the entire width. A guide duct 28, in which the form-fit element 38 of the valve unit 30 is guided, is formed within the valve sleeve 20. The maximum open position, with the form-fit element 38 being at the detent on the end of the guide 28, is illustrated in the illustration on the right in FIG. 10. It can be seen in FIG. 11 that the front end of the central member 47 terminates shortly before the end of the access opening 37 of the cavity 31 of the valve unit 30. The hydraulic fluid which flows in radially from the outside is diverted through in the direction towards the diffusor insert 40 as a result of the sleeve wall opening 24 being congruent with the passage opening 34, the hydraulic fluid in said diffusor insert 40 impacting the curved surface between the partition walls 44, 45, and then being diverted in the direction towards the outlet 3. It is achieved by the complete hydraulic separation by the partition walls 44, 45, 46 that the partial flows of hydraulic fluid entering through the passage openings 34 directly meet one another, wherein the partition walls 44, 45, 46 preferably extend up to the end of the passage openings 34.

A longitudinal sectional view of the adjustment valve 10, having the available flow path, is illustrated in FIG. 12. The hydraulic fluid enters the cavity 31 radially from the outside through the sleeve wall openings 24 and the passage openings 34, and is deflected on the surface of the diffusor insert 40 and diverted by 90° in the direction towards the outlet 3.

A cross-sectional view of the adjustment valve 10 according to FIG. 9 in the assembled state is shown in FIG. 13. The three sleeve wall openings 24, 25, 26 are uniformly distributed across the circumference, said sleeve wall openings 24, 25, 26 in turn being fluidically connected to the flow duct, or the inlet, respectively. Hydraulic fluid flows through the sleeve wall openings 24, 25, 26 and the correspondingly aligned passage openings 34, 35, 36 of the valve unit 30 into the cavity 31. The partial flows of the hydraulic fluid impact the valve unit 40 and are prevented from directly meeting one another by the partition walls in the region of the inflow. The ducts that are formed by the partition walls converge in the direction towards the outlet 3 in such a way that a common fluid flow of the hydraulic fluid is present at the access opening 37.

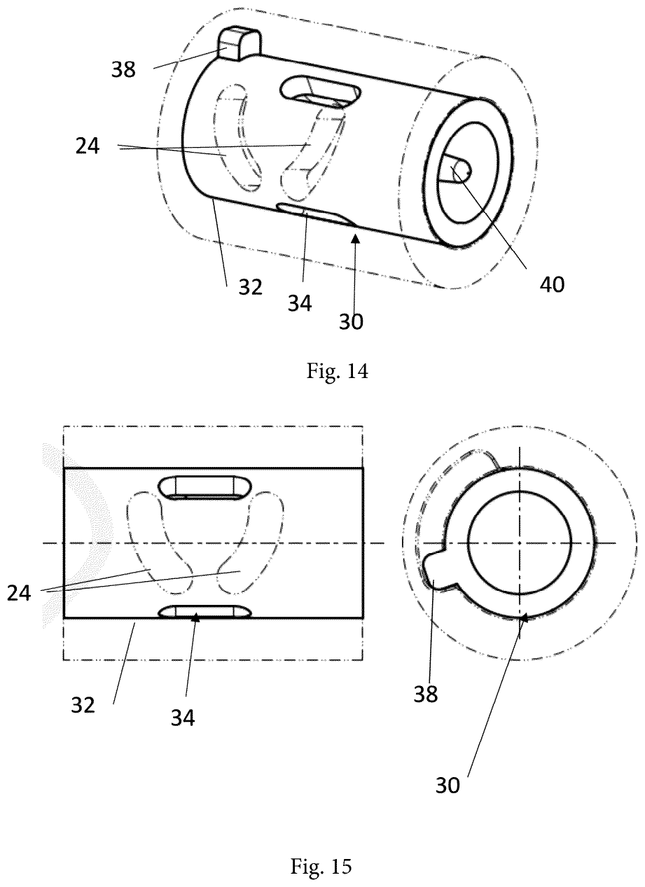

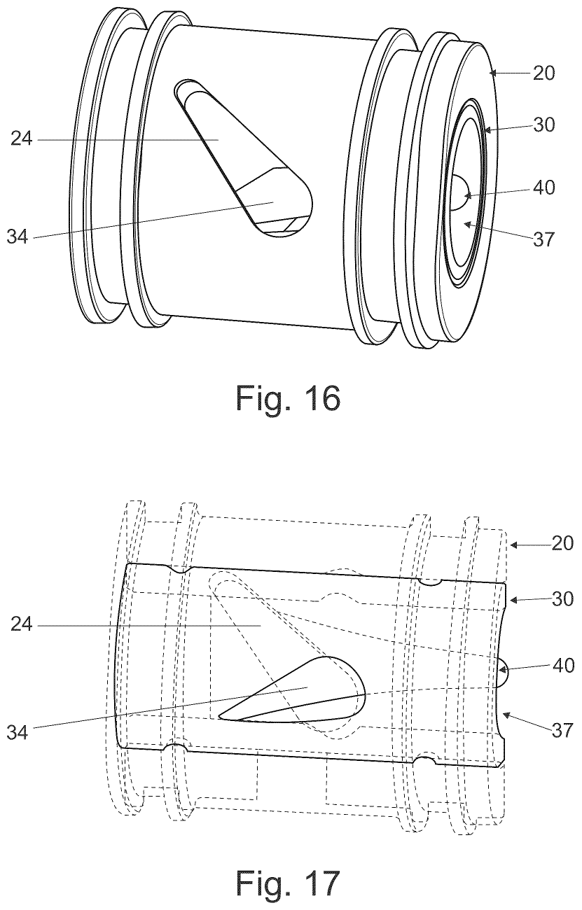

The adjustment valve is shown in a closed position in FIGS. 14 and 15. The construction corresponds substantially to that of FIGS. 11 and 10. It can be seen in FIG. 15 that the form-fit element 38 within the guide in the valve sleeve is rotated up to the opposite detent. As a result, the valve unit 30 is moved from the open position to the closed position illustrated, in which there is no congruence whatsoever between the sleeve wall opening 24 and the passage openings 34. Hydraulic fluid cannot flow from the outside through the sleeve wall opening 24 and through the passage openings 34, with the exception of any potential leakage which can be minimized or precluded by minimizing the gap widths.

A variant of the adjustment valve 10 is illustrated in FIG. 16; here too, a valve sleeve 20 having a valve unit 30 which is disposed so as to be rotatable therein, and a diffusor insert 40 which is disposed in a rotationally fixed manner and axially fastened therein, is illustrated. The differences in comparison to the preceding embodiments relate to the geometry and embodiment of the sleeve wall openings 24 and of the passage openings 34. Here too, three sleeve wall openings and three passage openings are provided; alternative embodiments having only two sleeve wall openings and passage openings, or more than three sleeve wall openings and passage openings, and optionally only one sleeve wall opening and one passage opening, are likewise possible.

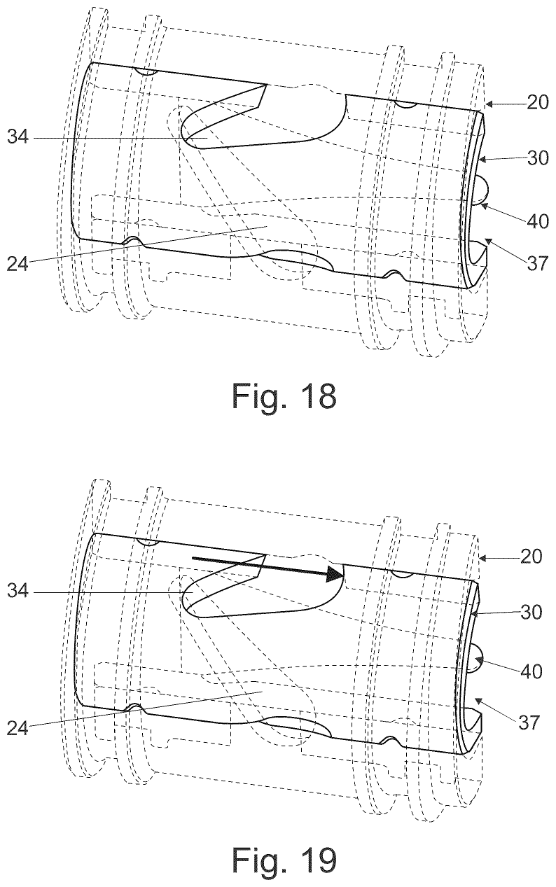

In the exemplary embodiment illustrated, the sleeve wall openings 24 are designed as slots which are formed so as to be inclined in relation to the longitudinal extent of the adjustment valve within the wall of the valve unit 30 and the valve sleeve 20. The inclination of the sleeve wall openings 24 and the inclination of the passage openings 34 are counter to one another so that the slots intersect in the course of a rotation from the open position illustrated to the closed position illustrated in FIGS. 18 and 19. In the embodiment illustrated, the width of the slots is continuously enlarged from the rearward end towards the front end facing the access opening 37, because the lateral flanks of the slots are not oriented parallel to one another, but diverge from back to front in the direction towards the outlet.

In FIGS. 18 and 19, the valve sleeve 20 and the valve insert 30 are in an almost closed position. The available passage cross section, which results from the congruence of the sleeve wall openings 24 and the passage openings 34, is reduced in comparison to the adjustment according to FIG. 16. The position at which the hydraulic fluid enters the cavity 31 of the valve unit 30 moves from approximately the centre of the valve unit 30 to the rear end, in the direction towards the shoulder of the diffusor insert that bears on the internal side of the cavity 31 of the valve unit 30. The arrow in FIG. 19 indicates the direction of movement of the available passage cross section into the cavity 31 when the valve unit 30 is rotated downwards. In this instance, the available passage cross section moves from the rear position downwards and forwards. Owing to this repositioning of the available passage cross section towards the front, the path of the hydraulic fluid from the passage openings to the surface of the diffusor insert 40 is enlarged in such a way that this results in an optimized diversion. A reduced volumetric flow owing to the smaller passage cross section, as is illustrated in FIGS. 18 and 19, can enter the cavity closer to the surface of the diffusor insert 40, and can be directed through the duct between the partition walls in the direction towards the outlet.

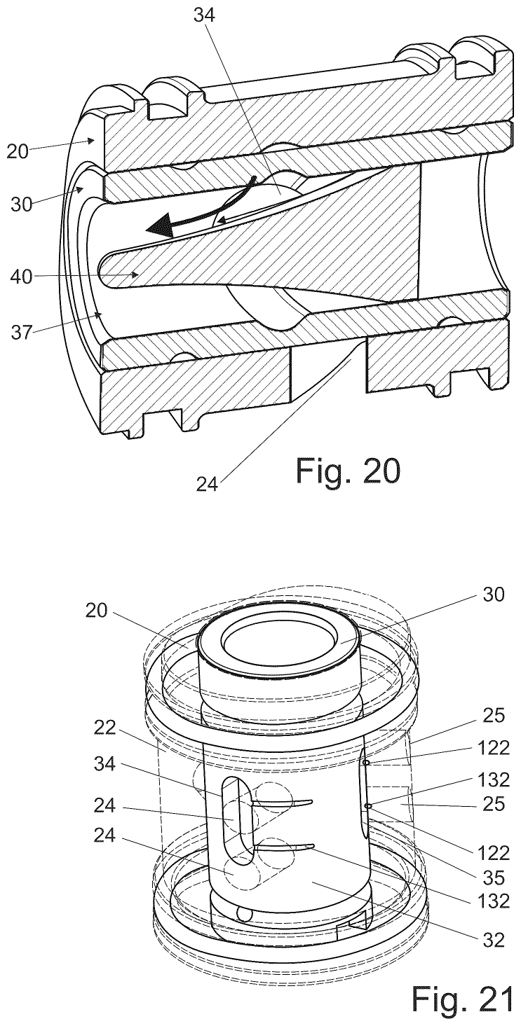

The embodiment of FIGS. 16 to 19 is shown in a cross-sectional illustration in FIG. 20. The sleeve wall opening in the valve sleeve 20 is not shown; the passage opening 34 moves relative to the sleeve wall opening during corresponding rotation, wherein the available passage cross section of the superimposed sleeve wall openings and passage openings moves in the circumferential direction as well as in the axial extent. In the position according to FIGS. 18 and 19, the hydraulic fluid enters the cavity 31 further back at the rear end, which is indicated by the longer rear arrow. In the open position according to FIGS. 16 and 17, a larger volumetric flow passes through the available passage cross section of the sleeve wall openings and passage openings, which is indicated by the front, thicker arrow.

As a result of the adjustment or rotation of the congruence of the sleeve wall opening and the passage opening, the available passage cross section, and thus the inflow of the hydraulic fluid into the cavity, moves along the longitudinal extent in the direction towards the diffusor insert 40. The axial position of the available passage cross section and the position of the inflowing hydraulic fluid vary as a function of the relative position of the valve sleeve 20 and the valve unit 30. The diffusor insert 40 herein is shaped in such a way that a fluidic optimum is achieved in such a way that no turbulent flow, or only a minor turbulent flow, is present in the inflow into the adjustment valve.

The adjustment valve having the valve sleeve 20 and the valve unit 30 disposed therein is illustrated in a partially transparent view in FIG. 21. Here too, the valve unit 30 is of a substantially cylindrical design and rotatably mounted in the valve sleeve 20. Two sleeve wall openings 24 are disposed below one another in the sleeve wall 22, so as to be mutually spaced apart in the longitudinal extent or axial extent. A total of three pairs of sleeve wall openings 24 in the form of bores which are disposed below one another are distributed at 120° about the circumference of the valve sleeve 20; the second pair of sleeve wall openings 25 can still be seen on the right side. In a manner corresponding to the embodiment of the adjustment valves of the previous figures, passage openings 34, 35 are likewise formed on the valve unit wall 32 so as to be distributed at 120° about the circumference, in the exemplary embodiment illustrated as a straight slot. In one embodiment, the hydraulic fluid flows from the sleeve wall openings 24, 25 radially towards the inside, through the passage openings 34, 35, into the cavity within the valve unit 30, and from there into the connected flow duct. A diffusor is optionally disposed in the cavity. Irrespective of a diffusor being disposed in the cavity, ducts 132 which have a semi-circular cross section and extend in a mutually parallel manner in the circumferential direction away from the passage opening 34, 35, are incorporated on the external side of the valve unit wall 32. The ducts 132 are formed as ducts which are open towards the outside and are closed by the sleeve wall 22. The length in the circumferential direction is sized in such a way that the slot-type passage opening 34 can be rotated to the extent that the sleeve wall openings 24 are no longer fluidically connected to the ducts 132, but also are not in contact with the subsequent passage opening 35, when the valve unit 30 is rotated relative to the valve sleeve 20. The spacing between the duct 132 and the passage opening not connected thereto is so large that the sleeve wall opening 24 lies opposite a smooth valve unit wall 32. In the embodiment of FIG. 21, channels or ducts 122 are likewise incorporated in the valve sleeve 20, which are open towards the inside in the sleeve wall 22 and are designed in a manner corresponding to the ducts 132 of the valve unit 30. The channels or ducts 122, 132 preferably have a substantially constant width and/or depth, so that a laminar flow is formed in the potentially assembled duct.

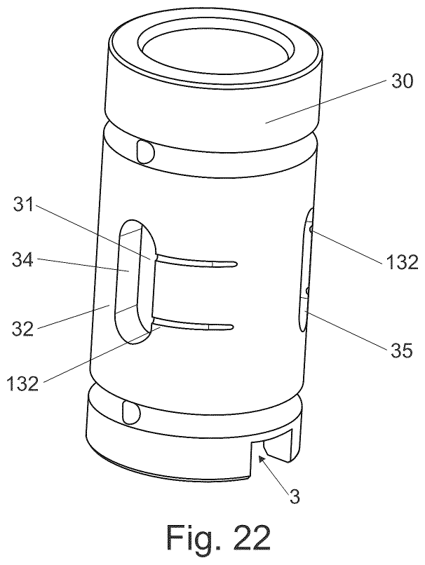

The valve unit 30 of the adjustment valve according to FIG. 21 is shown in an individual illustration in FIG. 22. The valve unit 30 is designed as a bushing or sleeve, and has a valve unit wall 32, grooves for receiving annular seals being disposed in the external side of the latter. Passage openings 34, 35 which reach up to a cavity 31 within the valve unit 30 are likewise incorporated in the valve unit wall 32. The upper side of the valve unit 30 is closed; the cavity 31 opens into an outlet 3 on the lower side, said outlet 3 in turn leading to a flow duct as described above. In a reversed flow direction, thus in a flow direction in which the hydraulic fluid first enters the cavity 31 and is directed through the passage openings 34, 35 to the sleeve wall openings, the access opening is an inlet.

It can be seen in FIG. 22 that the ducts 132 only extend across a partial circumference of the external side of the valve unit wall 32. The ducts open into the passage opening 34, 35 on one side, wherein the cross section, or the depth, of the duct 132 is enlarged in the direction towards the passage opening 34, 35. The enlargement ideally takes place without steps in order to avoid turbulent flows. Deviating from the rectilinear embodiment of the ducts 132 about the circumference of the external side of the valve unit wall 32 illustrated, these ducts may also perform a bend in the axial extent in such a way that the ducts 132 do not run at the same height level of the valve unit 32.

As a result of the ducts 132 directed in the direction towards the passage openings 34, 35 it is possible to provide a throttle geometry which ensures laminar throttling. Flow noises are reduced, and intense local heating at the throttle point in the adjustment valve minimized, as a result. The ducts 132 can also be disposed in all other shapes and configurations of passage openings and sleeve wall openings described above. If the ducts 132 are disposed in the valve unit 30, the passage openings 34, 35 are complemented by these ducts 132. In the case of a not completely open adjustment valve, and a supply of the hydraulic fluid through the valve sleeve 20, for example, the hydraulic fluid at least partially impacts the ducts 132 in such a way that the hydraulic fluid has to increasingly cover a longer distance in order to reach the respective valve exit. The characteristic of this flow path is laminar. Alternatively or additionally, the ducts 122 or ducts 122 are formed or disposed on the internal side of the sleeve wall 22, which ensures that the flow is correspondingly pacified, or a turbulent flow is avoided, when the flow direction is reversed, for example.

1. Adjustment valve for a hydraulic actuator in a flow duct of an orthopaedic device, comprising:
a valve sleeve;
a valve unit;
wherein the valve sleeve comprises
a sleeve wall which forms a receptacle space, and
at least one radially aligned sleeve wall opening formed in the sleeve wall;
wherein the valve unit is disposed within the receptacle space of the valve sleeve so as to be repositionable between a closed position and an open position, and wherein a cavity is formed by the valve unit;
wherein the valve unit comprises at least one valve unit wall which lies opposite the at least one sleeve wall opening of the valve sleeve, wherein at least one passage opening is formed in the at least one valve unit wall that in an open position lies opposite a corresponding sleeve wall opening of said at least one sleeve wall opening; and
at least one duct formed in at least one of the sleeve wall and the at least one valve unit wall, wherein if the at least one duct is formed in the sleeve wall the at least one duct opens out at the at least one radially aligned sleeve wall opening, and wherein if the at least one duct is formed on the at least one valve unit wall the at least one duct opens out at the at least one passage opening.

2. The adjustment valve according to claim 1, wherein the at least one duct comprises a first duct formed on the sleeve wall and a second duct formed on the at least one valve unit wall, wherein the first duct is opposite the second duct.

3. The adjustment valve according to claim 1 wherein the at least one duct is at least partially formed as a duct or channel which is open towards an outside the valve unit.

4. The adjustment valve according to claim 1 wherein the at least one duct is enlarged in a direction towards the at least one radially aligned sleeve wall opening and/or the at least one passage opening.

5. The adjustment valve according to claim 1 wherein the cavity formed by the valve unit is a closed design with the exception of the at least one passage opening and an access opening.

6. The adjustment valve according to claim 1 wherein the valve unit is mounted so as to be rotatable or displaceable in the valve sleeve.

7. The adjustment valve according to claim 1 wherein the valve unit is coupled to an adjustment device.

8. The adjustment valve according to claim 1 wherein the valve sleeve is formed as part of a housing or as a separate component.

9. The adjustment valve according to claim 1 wherein the at least one radially aligned sleeve wall opening and/or the at least one passage opening are/is designed as a slot or bore.

10. The adjustment valve according to claim 9, wherein the slot is oriented obliquely in relation to a longitudinal extent of the adjustment valve.

11. The adjustment valve according to claim 9 wherein the slot is of a rectilinear or curved design, and/or wherein the slot varies in terms of a width along a longitudinal extent of the slot.

12. The adjustment valve according to claim 1 wherein the at least one duct comprises a plurality of ducts which are disposed or formed such that in the closed position there is no fluidic connection between the at least one radially aligned sleeve wall opening and the at least one passage opening.

13. The adjustment valve according to claim 1 wherein the cavity formed by the valve unit opens into an outlet, and further comprising a diffusor insert configured to deflect hydraulic fluid from the at least one passage opening) in a direction towards an outlet disposed in the cavity.

14. The adjustment valve according to claim 13, wherein the diffusor insert is made separately from the valve unit and is fastened to or in the valve unit, or wherein the diffusor insert is an integral constituent part of the valve unit.

15. The adjustment valve according to claim 13 wherein the at least one passage opening comprises a plurality of passage openings, and wherein the diffusor insert comprises partition walls which are disposed between at least two passage openings of the plurality of passage openings.

16. The adjustment valve according to claim 15, wherein the partition walls extend up to the outlet or terminate in front of the outlet.

17. The adjustment valve according to claim 13 wherein the diffusor insert is designed to taper in a direction towards the outlet.

18. The adjustment valve according to claim 3, wherein the at least one duct is configured such that a laminar flow is formed in the at least one duct, and wherein there is a proportional correlation between pressure and volumetric flow in the at least one duct.

19. The adjustment valve according to claim 3 wherein the at least one duct has a constant width and/or a constant depth.
